(19) United States
(12) Patent Application Publication
(10) Pub. No.: US 2007/0243597 A1
(43) Pub. Date: Oct. 18, 2007

(54) Disinfectant Solutions

(75) Inventors: Robert Kenyon, Surrey (GB); Nicholas Meakin, Suffolk (GB)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: Forum Bioscience Holdings Limited, Surrey (GB)

(21) Appl. No.: 10/594,374

(22) PCT Filed: Apr. 1, 2005
(86) PCT No.: PCT/GB05/01261

§ 371 Date: Jun. 13, 2007

(30) Foreign Application Priority Data
Apr. 1, 2004 (GB) 0407478.7

(51) Int. Cl. C11D 3/20; A61K 33/14; C12N 7/06; C02F 1/461

(52) U.S. Cl. 435/238; 422/37; 424/661; 510/109; 510/161; 510/218; 510/379

(57) ABSTRACT

Embodiments disclosed herein relate to improved disinfectant solutions. In certain examples, the disinfectant solutions may be used to kill micro-organisms such as bacteria and viruses, for cleaning medical apparatus or for disinfecting other devices.

The present invention relates to improved disinfectant solutions, particularly for killing micro-organisms such as bacteria and viruses. Whilst many different types of disinfectants are already known, these are generally harmful to animals and contact with them should be avoided. Furthermore, conventional disinfectants are harmful to the environment and their use should therefore be kept to a minimum. The present invention relates to disinfectant solutions which are safe and which do not harm the environment. Therefore, the disinfectants solutions of the invention may be used freely and can even be safely ingested by animals, including humans.

The ideal disinfectant is one that works against a variety of organisms, such as bacteria, bacterial spores, protozoa, fungi and viruses. Furthermore, it is beneficial for the disinfectant to work in any environment and to be non-toxic, non-irritating, non-corrosive and relatively inexpensive, as disinfectants are frequently used in large quantities. Unfortunately, none of the known disinfectants is ideal and so the disinfectant has to be carefully chosen to best suit the situation in which it is to be used.

Chlorhexadine disinfectants are relatively non-corrosive and nonirritating. They do maintain their effectiveness in the presence of some organic material but will precipitate out in hard water. While they have a relatively broad spectrum of activity, chlorhexadine disinfectants are not very effective against some viruses (such as those that cause foot-and-mouth disease) and bacteria (such as those than cause tuberculosis and Johne's disease). Examples of commercially available chlorhexadine disinfectants include Nolvasan® and Virosan®.

Disinfectants containing hypochlorite are effective against a large variety of organisms (including those responsible for foot-and-mouth disease and bovine tuberculosis). Household bleach (for example Chlorox®) contains 5.25 to 6% sodium hypochlorite and is readily available and cheap. However, hypochlorite disinfectants have the disadvantage that they can be irritating, they can damage clothing and they ate corrosive to equipment. They also quickly become inactive in the presence of organic material.

Phenols are broad-spectrum disinfectants but they are not effective against non-enveloped viruses (like the foot-and mouth disease virus). The fact that phenols maintain their activity in the presence of organic material makes them useful for use in livestock operations. Although phenols ate relatively non-toxic, prolonged skin exposure can be irritating. Examples of commercially available phenolic disinfectants include One-Stroke Environ® and Lysol®.

Oxidising agents are peroxide-based and they include the commonly used wound disinfectant hydrogen peroxide. These agents are broad spectrum disinfectants and are generally effective against diseases such as foot-and-mouth and tuberculosis. They are inactivated by the presence of organic material. Oxidising agents ate relatively safe in their diluted forms, but they may be irritating and they can damage clothing in their concentrated forms. Examples of commercially available oxidising disinfectants include Trivectant®, Virkon® and OxySept 333®.

Iodine-based disinfectants are also broad-spectrum in their activity. They ate often formulated with soaps to form products such as surgical scrubs. They are inactivated by organic material and, although they are relatively safe, concentrated forms (such as tincture of iodine) can be irritating and can stain clothes. Examples of commercially available iodine disinfectants include Betadyne® and Povidone®.

Finally, quaternary ammonium disinfectants contain ammonium. These disinfectants are ineffective against non-enveloped viruses and ate inactivated by organic material, hard water and soap. Examples of quaternary ammonium disinfectants available commercially include Roccal-D® and Zepharin®.

In recent years, the environmental impact of disinfectants has also become particularly important. Many of the commonly used disinfectants, such as household bleach, have an extremely high chemical load and therefore ate potentially very harmful to the environment. There is the urgent need for a disinfectant which has a low chemical load and therefore will have a reduced impact on the environment. The chlorine content of disinfectants in particular has come under scrutiny in Europe.

In light of the foregoing, it is clear that whilst there are a large number of different types of disinfectants available, there remains a need for a disinfectant which is effective against all types of bacteria, viruses, etc, which can be used in all environments (including in the presence of organic material), which is safe and non-corrosive, which is also cheap and widely available and which has a low chemical load and preferably a low chlorine content.

In accordance with a first aspect of the present invention, a disinfectant comprising electrochemically activated water with a low chlorine content is provided.

Electrochemically activated water (also sometimes referred to as hydroactive water) is water which has undergone electrochemical activation (ECA). Such treatment involves the exposure of water and the natural salts therein or salts added to it, to a substantial electrical potential difference.

If one places an anode (+) and a cathode (−) in pure water and applies a direct current, electrolysis of the water will occur at the poles leading to a breakdown of the water into its constituent elements, producing gaseous hydrogen and oxygen. However, if sodium chloride is added to the water to form a solution, the dominant electrolysis end product is hypochlorite or hypochlorous acid (HOCl), a chlorine-based reagent which may be used to kill micro-organisms.

The electrochemical activation process is improved by interposing an ion-permeable membrane between the positive and negative electrodes, forming an anode chamber and a cathode chamber. Preferably, the aqueous sodium chloride solution is fed into both the anode chamber and the cathode chamber and the sodium chloride, which is in its ionised form in solution (Na⁺ and Cl⁻) is exposed to the controlled electrical potential difference between the cathode and the anode. This potential difference causes the Na⁺ ions to migrate to the cathode and the Cl⁻ ions to migrate to the anode. The membrane which separates the anode chamber and the cathode chamber allows ions to pass unimpeded, whilst the un-ionised water and any organic molecules in the water are unable to pass through the membrane.

The presence of an ion-permeable non-ceramic membrane in the electrolysis apparatus allows the necessary ions to be concentrated in the anode and cathode chambers, which results in the formation of metastable ions with high biocidal activity and very low chlorine levels. Although a similar process takes place in conventional electrochemical activation processes, the presence of anion-permeable membrane prevents the complex reactive species formed at the cathode and anode from reacting with one another and being neutralised. The specific choice of a non-ceramic membrane further refines the chemical processes.

As the electrical potential is applied, high concentrations of Cl⁻ and OH⁻ build up on the anode side of the membrane and Na⁺ and H⁺ build up on the cathode side of the membrane. The unstable chemical state results in complex reactions which produce a metastable solution containing a wide variety of very reactive ions and molecules, such as those set out in Table 1.

TABLE 1

| | Reactive Molecules | Reactive Ions |
|---|---|---|
| Anolyte | O₃ | |
| | O₂ | |
| | H₂O₂ | OH⁻ |
| | ClO₂ | ClO⁻ |
| | HClO | |
| | Cl₂ | |
| | HCl | |
| | HClO₃ | |
| Catholyte | H₂O₂ | H₃O⁺ |
| | NaOH | Na⁺ |
| | H₂ | |

It is the formation of these complex chemical species which leads to the formation of solutions described as electrochemically activated water or hydroactive water. Some of the more important reactive constituents formed during the electrochemical activation of the sodium chloride solution include hypochlorite (HClO), hydrogen peroxide (H₂O₂), ozone (O₃), chlorine (Cl₂) and chloric acid (HClO₃). Most of these compounds are formed in the anode chamber. They are acidic, giving the anolyte a pH of between 2.4 and 4 and oxidising activity. In the cathode chamber, the reactive species are basic and are reducing agents. The catholyte will have a pH of between 10 and 12.

The anolyte and catholyte produced by the electrochemical activation of an aqueous sodium chloride solution also exhibit opposing potentials, the anolyte having a redox potential of +1050 mV, while the catholyte has a redox potential of −850 mV. This is compared to the redox potential of the starting material of approximately +300 to 400 mV.

In the apparatus with an ion-permeable membrane, two distinct solutions are formed, the catholyte and the anolyte, and these solutions can be separately extracted or they can be mixed.

The anolyte solution exhibits mild oxidative power and can destroy micro-organisms, and therefore has useful sterilizing and disinfectant properties. However, the use of such anolyte solutions is safe. Whilst the anolyte solutions are capable of killing bacteria and viruses, etc., the freshly prepared solutions are benign in terms of fumes, corrosion and their effect on the skin of humans and animals.

The catholyte has properties which make it useful as a detergent and as a surfactant. Its reducing power also mean that the catholyte is effective in precipitating metal ions out of water and it can be used to soften hard water.

These properties of the anolytes and catholytes of the electrochemically activated water have previously been identified but only when the solution has been produced with high residual hypochlorite content of greater than 50 ppm. It has now unexpectedly been found that electrochemically activated water with a low chlorine content also exhibits the disinfectant properties, despite containing only little, if any, hypochlorite when generated using non-ceramic membranes.

TABLE 2

| | | | Chlorine |
|---|---|---|---|
| Current | O.R.P. | pH | content (ppm) |
| 3 | 1052 | 2.67 | 35 |
| 4 | 1086 | 2.67 | 58 |
| 5 | 1104 | 2.58 | 95 |
| 6 | 1107 | 2.39 | 103 |
| 7 | 1108 | 2.39 | 129 |
| 8 | 1111 | 2.28 | 134 |
| 9 | 1112 | 2.23 | 156 |
| 10 | 1114 | 2.19 | 198 |
| 11 | 1115 | 2.15 | 221 |
| 12 | 1115 | 2.11 | 226 |
| 13 | 1115 | 2.11 | 230 |
| 14 | 1115 | 2.1 | 249 |
| 15 | 1115 | 2.07 | 258 |
| 16 | 1115 | 2.04 | 295 |
| 17 | 1115 | 2.02 | 371 |

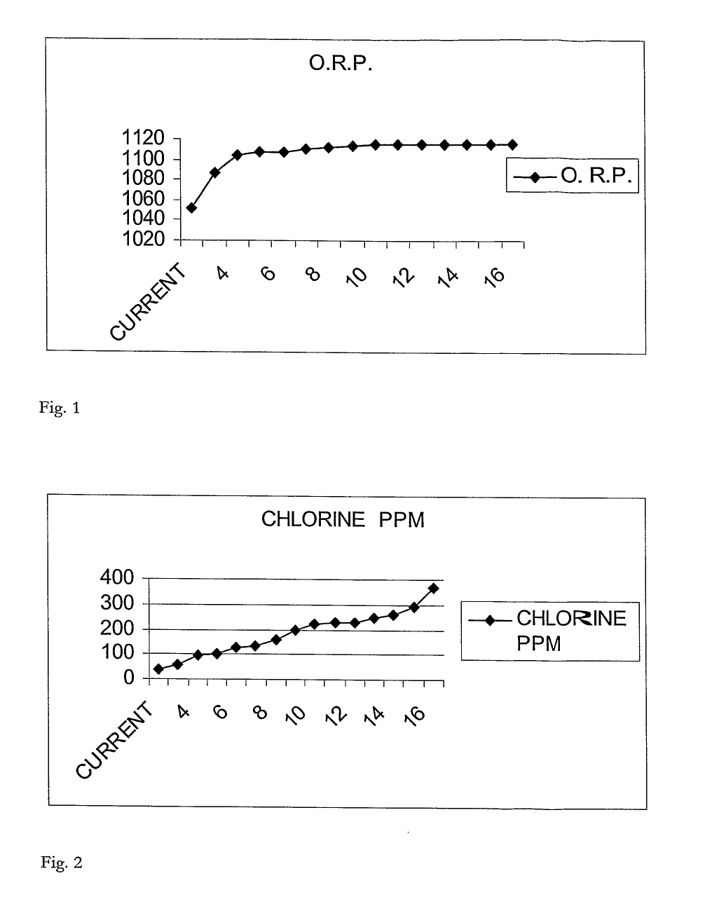

FIG. 1 is a graph indicating the ORP of electronically activated water in mV versus the electrical current used in its production for an ECA 2000 machine.

FIG. 2 is a graph indicating the increase in chlorine in ppm with current for ECA 2000 electronically activated water generator.

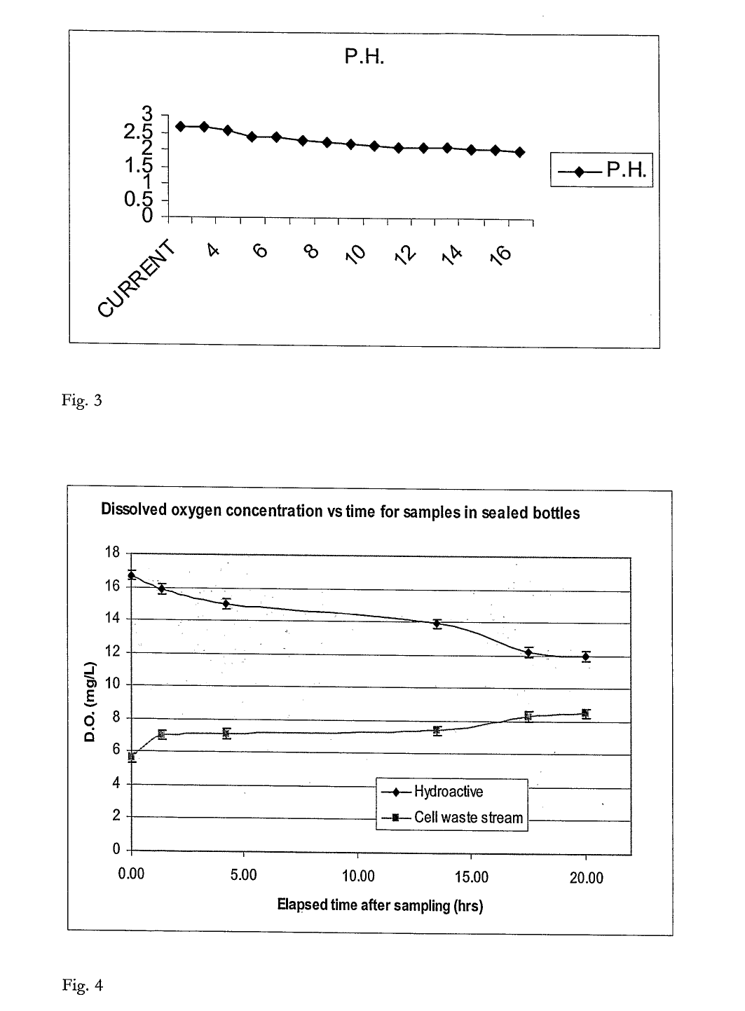

FIG. 3 is a graph showing the variation of pH with current for ECA 2000 electronically activated water generator.

It is generally believed that the disinfectant activity of electrochemically activated water is a function of the hypochlorite concentration of the water. As the concentration of hypochlorite increases, so does the redox potential (ORP) and the disinfectant activity of the electrochemically activated water. As a result, it is generally considered that the higher the hypochlorite content of the electrochemically activated water, the greater its disinfectant activity. Indeed, it is generally thought that the electrochemically activated water must include at least 50 to 200 ppm hypochlorite in order for it to exhibit acceptable disinfectant activity. Where electrochemically activated water has been used in the past as a disinfectant, the hypochlorite concentration has been greater than 50 ppm and is usually much greater, sometimes as high as 650 ppm.

Therefore, it was previously believed that electrochemically activated water having only chlorine content of less than 8 ppm would not exhibit any disinfectant activity and would not be effective against micro-organisms and certainly not effective against resilient viruses.

However, as can be seen from Table 2 the ORP of solutions produced on an ECA 2000 generator starts to plateau at 3-4 amps and from 6-8 amps onwards the figure remains almost constant whereas the chlorine content climbs rapidly. If the ORP is a measure of the disinfectant activity then the graphs of FIGS. 1, 2 and 3 and Table 2 are indicative that electrochemically activated water solutions, produced on an ECA 2000 machine, should be effective disinfectants at total chlorine levels of less than 35 ppm (see Table 2).

This hypothesis has now been demonstrated. That is, when the electrochemically activated water produced using the above rationale to minimise the chlorine content is diluted with mains water to a chlorine content of less than 8 ppm it still exhibits disinfectant activity.

The disinfectant activity of this water is not a result of the hypochlorite in the water, but rather it appears to be due to the presence of an activated chlorine and oxygen species. This activity is thought to be “masked” in conventional electrochemically activated water by the levels of and the activity of the hypochlorite. It has now been found that the activated chlorine and oxygen species produces a redox potential high enough for the water to have disinfectant activity (greater than +900 mV), whilst having a minimal chlorine content.

It is thought that the active oxygen species in the electrochemically activated water is highly reactive, reacting with many organic compounds including olefins, dienes, sulphides, aromatics, hetero-aromatics, terpenes, steroids, fatty acids, flavones, tetracyclines, vitamins, amino acids, proteins, nucleic acids, blood and bile pigments.

The activity of this minimal chlorine electrochemically activated water against bacteria, viruses and fungi species can be seen from Tables 3, 4 and 5 below.

TABLE 3
Anti Microbial Efficacy of Electronically Activated Water with
Minimal Chlorine content as produced by ECA 2000 generator
Dilution-neutralisation method for 1 minute contact time.

| | Viable count (cfu/ml) for test mixture (Nₐ) at concentrations: | | |
|---|---|---|---|
| | Neat | 50% v/v | 5% v/v |
| Test organism | (approx 140 ppm) | (approx 70 ppm) | (approx 7 ppm) |
| Pseudomonas | <1.5 × 10² | <1.5 × 10² | <1.5 × 10² |
| aeruginosa | | | |
| Staphylococcus | <1.5 × 10² | <1.5 × 10² | >3.0 × 10³ |
| aureus | | | |
| Salmonella | <1.5 × 10² | <1.5 × 10² | <1.5 × 10² |
| (enteritidis) abony | | | |
| Escherichia coli | <1.5 × 10² | <1.5 × 10² | <1.5 × 10² |
| Campylobacter | <1.5 × 10² | <1.5 × 10² | <1.5 × 10² |
| jejuni | | | |

| | Reduction in viability at test concentration: | | |
|---|---|---|---|
| Test organism | Neat | 50% v/v | 5% v/v |
| Pseudomonas | 2.6 × 10⁵ | 2.6 × 10⁵ | 2.6 × 10⁵ |
| aeruginosa | | | |
| Staphylococcus | 3.3 × 10⁵ | 3.3 × 10⁵ | 1.7 × 10⁴ |
| aureus | | | |
| Salmonella | 3.3 × 10⁵ | 3.3 × 10⁵ | 3.3 × 10⁵ |
| (enteritidis) abony | | | |
| Escherichia coli | 3.1 × 10⁵ | 3.1 × 10⁵ | 3.1 × 10⁵ |
| Campylobacter | 1.3 × 10⁵ | 1.3 × 10⁵ | 1.3 × 10⁵ |
| jejuni | | | |

TABLE 4
Electrochemically Activated Water produced by an
ECA 2000 generator: Virucidal efficacy against Influenza
A virus: Reduction in virus infectivity

| | Reduction in Infectivity (log₁₀)/Contact time | | | |
|---|---|---|---|---|
| | (minutes) | | | |
| Sample | 1 | 5 | 10 | 30 |
| Test substance Neat | ≧5.6 | ≧5.5 | ≧5.7 | ≧5.8 |
| Test substance | 3.7 | 4.2 | 4.9 | ≧5.8 |
| (approx 14 ppm Cl) | | | | |
| Test substance | 0.3 | 1.0 | 1.3 | 1.6 |
| (approx 1.5 ppm Cl) | | | | |
| Test substance | 0 (−0.2) | 0 (−0.1) | 0.4 | 0.4 |
| (approx 0.6 ppm Cl) | | | | |

TABLE 5
Measurement of inhibition of growth of Saprolegnia parasitica
by electrochemically activated water with 24 hour
incubation, 1 minute contact time.

| Hydroactive | Colony diameter (mm) | | | Mean | |
|---|---|---|---|---|---|
| concen- | Repli- | Repli- | Repli- | colony | % |
| tration | cate | cate | cate | diameter | Inhibition |
| (%) | A | B | C | (mm) | of growth |
| 0 | 36.30 | 34.55 | 35.25 | 35.37 | N/A |
| (SDW | | | | | |
| control) | | | | | |
| 100 | 33.30 | 32.60 | 28.40 | 31.43 | 11.14 |
| (Neat) | | | | | |
| 50 | 32.95 | 27.80 | 29.40 | 30.05 | 15.04 |
| 25 | 33.35 | 33.45 | 32.30 | 33.03 | 6.62 |
| 10 | 34.25 | 32.15 | 34.70 | 33.70 | 4.72 |
| 5 | 33.50 | 34.40 | 34.35 | 34.08 | 3.65 |
| 1 | 34.80 | 35.20 | 33.80 | 34.60 | 2.18 |

The activity of the electrochemically activated water has been demonstrated by the inventors to require the presence of a chlorine based species. As shown in table 6, a starting solution of sodium chloride was found to be necessary in order to achieve an anti-microbial activity.

TABLE 6
Electrochemically Activated Water pH, dissolved oxygen
content and antimicrobial efficacy - (10 minute challenge
against Escherichia coli.) produced from a range of inorganic
salts and acids at constant ionic strength.

| | Sodium | Sodium | Ace- | Cit- | Sodium | Sul- | |
|---|---|---|---|---|---|---|---|
| | Bicar- | di- | tic | ric | chlo- | phuric | Control |
| Salt | bonate | acetate | acid | acid | ride | acid | (water) |
| pH of | 8.8 | 4.9 | 3 | 2.1 | 3.3 | 1.41 | 7 |
| Anolyte | | | | | | | |
| Dissolved | >20 | 15.7 | 10.7 | 14.6 | 16-19 | 15.4 | 8-9 |
| Oxygen | | | | | | | |
| cfu/ml × | 1.5 | 1.7 | 0.23 | 6.4 | 0.000 | 0.000* | 1.9 |
| 10⁶ | | | | | | | |

(Sulphuric and acetic acid were demonstrated to have an antimicrobial effect by virtue of their acidity alone). In order to assess whether a chlorine based specie was required, citric acid was added to neat electrochemically activated water. Citric acid deactivates chlorine, as can be seen on Table 7.

TABLE 7
Effect of Citric Acid concentration on chlorine species present
in Electrochemically Activated Water (generated by ECA 20OO
equipment). As measured by ion chromatography.

| | | Percent of added | | | | |
|---|---|---|---|---|---|---|
| | | citric acid | | | | |
| | | 0.0% | 0.1% | 0.5% | 100% | |
| Chloride | Cl | 3223 | 3256 | 3216 | 23 | ppm |
| Hypo- | ClO− | ND | ND | ND | ND | ppm |
| chlorite | | | | | | |
| Chlorite | ClO2− | <1 | <1 | <1 | <1 | ppm |
| Chlorate | ClO3− | 0.3 | 0.2 | 0.2 | 0.3 | ppm |
| Per- | ClO4− | <10 | <10 | <10 | <10 | ppb |
| chlorate | | | | | | |
| Free | Cl2 | 30 | 20 | 0.0 | 0.0 | ppm |
| Chlorine | | | | | | |
| pH of | | 3.33 | 2.15 | 1.90 | 2.10 | |
| Anolyte | | | | | | |
| Dissolved | | 14.8 | 15.5 | 16.6 | 14.6 | mgs/l |
| Oxygen | | | | | | |
| Cfu/ml at | | 0.0000 | 4.3 | 0.95 | 3.0 | ×10⁶ |
| neat concen- | | | | | | |
| tration | | | | | | |

ND = Not Determined

As it can be seen from Table 7, as the level of chloride decreases, the anti-microbial effect decreases. Levels of hypo-chlorite, chlorite, chlorate and perchlorate remain constant.

Additionally, the level of chlorine dioxide produced according to a preferred embodiment of the invention has been determined. It was found that very little or no chlorine dioxide was produced. As shown on Table 8 and FIG. 7, chlorine dioxide was found to be minimal or absent in the electrochemically activated water.

TABLE 8
Electrochemically Activated water test results from sweep of
AC voltage across cell vs. current and dissolved. Chlorine Dioxide
concentration at 0.19M Sodium Chloride concentration.

| | | Sample Number | | | | |
|---|---|---|---|---|---|---|
| | Reference | 1 | 2 | 3 | 4 | 5 |
| AC Voltage across cell | | 7.9 | 12.75 | 29.6 | 47 | 45 |
| Current (Amps) | | 1.2 | 2.1 | 9 | 27 | 20 |
| Acidity pH | | 4.67 | 2.77 | 2.32 | 2.25 | 2.6 |
| ORP mV | 812 | 1045 | 1165 | 1189 | 1183 | 1202 |
| Dissolved Oxygen ppm | 8.7 | 11.3 | 15.7 | >20 | >20 | >20 |
| Chlorine Dioxide | 0 | 0 | 0 | <1 | <1 | 0 |
| Free Chlorine in gas above sample | 0 | 0 | 0 | 100 ppb | 1000 ppm | ND |

Qualitative chlorine dioxide was measured using UV/Visible spectrometer against chlorine dioxide standard, see FIG. 7.

FIG. 7 demonstrates the UV/visible absorption of Chlorine Dioxide vs Hypochlorous acid and Electrochemically Activated Water generated by ECA 2000 equipment. This enables approximate measurement of Chlorine Dioxide concentration.

The traces in FIG. 7 are:

| | |
|---|---|
| Trace (1) | chlorine dioxide |
| Trace (2) | chlorine dioxide at 1 ppm |
| Trace (3) | Hypochlorous acid |
| Trace (4) | 1:1 mixture of chlorine dioxide and hypochloric acid |
| Trace (5) | Sample 4 - shows only Hypochlorous acid (no chlorine |
| | dioxide) |
| Trace (6) | Sample 3 shows only Hypochlorous acid |

From FIG. 7 it can be determined that there is minimal chlorine dioxide produced when the ECA 2000 generator is used to produce electrochemically activated water.

Thus, it is unlikely that the anti-microbial activity is due to chlorine dioxide in the electrochemically activated water.

Additionally, the electrochemically active water of the invention is thought to contain minimal or no ozone when the ECA generator is used.

Therefore, according to a first aspect of the present invention, a disinfectant composition is provided, comprising the anolyte of electrochemically activated water with minimal chlorine content.

It is desirable to have electrochemically activated water with a minimal chlorine content for a number of reasons. The water is so benign that, at effective doses, it is safe for ingestion by humans and animals and fish. The water can be added to food or drinking water without having any harmful effect. The minimal chlorine content also means that ingestion of the water will not lead the subject to significantly increase or reduce its water intake. This is particularly beneficial where the water is fed to farm animals such as chickens. Increased water intake in birds leads (for example) to wet litter which is undesirable as wet litter is more likely to stick to the birds and cause burning. What is mote, the wet litter is more likely to provide a good breeding ground for micro-organisms, which in turn promotes the spread of disease. Where the chlorine content of the electrochemically activated water exceeds 120 ppm, it will kill birds if ingested.

In another embodiment of the present invention, the disinfectant comprises electrochemically activated water with a chlorine content of 0.1 to 10 ppm, more preferably it is 0.2 to 8 ppm and most preferably it is 0.3 to 6 ppm.

The minimal chlorine electrochemically activated water generated by the ECA 2000 equipment used in the present invention has a redox potential of at least +900 mV. Preferably, the electrochemically activated water used in the present invention includes an active oxygen species and the concentration of the active oxygen is preferably between 11 and 20 mg/l.

In a preferred embodiment, the level of chlorine dioxide (ClO₂) present in the electrochemically activated water is less than 10 ppm, preferably less than 5 ppm, and most preferably less than 1 ppm. For example, the level of chlorine dioxide is no more than 9, 8, 7, 6, 5, 4, 3, 2 or 1 ppm.

In a further preferred embodiment, the level of hypochlorite in the electrochemically activated water is less than 10 ppm, preferably less than 5 ppm, most preferably less than 1 ppm. For example, the level of hypochlorite is no more than 9, 8, 7, 6, 5, 4, 3, 2 or 1 ppm.

In another embodiment of the invention, the disinfectant comprises electrochemically activated water according to the invention, with the pH adjusted to a desired value or range. The disinfectant anolyte of the electrochemically activated water has a pH of below 4. However, in many applications of the disinfectant compositions, the pH is preferably higher than this. For example, in certain preferred embodiments, the pH of the composition is between 3 and 8, or between 4 and 7. It is also desirable for the pH to be kept at a constant pH, with variations of 0.5 of a unit or less, and preferably of 0.1 of a unit or less.

The compositions according to the present invention which have a stable pH value of 3-8 or 4-7 ate advantageous for use in physiological systems, as proposed herein.

The compositions of the present invention have the advantage that their pH may be adjusted without the loss of disinfectant and biocidal activity. In one embodiment, the pH of the anolyte of electrochemically activated water with a minimal chlorine content is adjusted by adding a amount of the catholyte of the electrochemically activated water sufficient to produce the desire pH value. This combination of the anolyte and the catholyte of electrochemically activated water retains the disinfectant and biocidal activity of the anolyte.

The electrochemically activated water with a minimal chlorine content according to the present invention can be produced in a number of ways. The chlorine content is affected by the amount of sodium chloride in the water prior to the electrochemical activation process, the current used to electrolyse the solution and the rate of flow of the salt solution through the different chambers of the electrolytic cell.

The amount of chlorine in the electrochemically activated water will be affected by the length of time the anolyte solution is exposed to the electrical current and the size of that current.

In order to produce the compositions according to the first aspect of the present invention, the electrolysis process must be run at a voltage and/or power density which is higher than that at which O₂ and Cl₂ are produced, but which is also lower than that at which HClO⁻ and ClO₂ are produced. Preferably, the voltage adjacent to the electrode face is between 1.35 and 1.63V, preferably between 1.4 and 1.5V and mote preferably between 1.42 and 1.47V. It is important to note that these values do not refer to the voltage across the whole system. The chemical reactions are taking place at the face of the electrodes and so it is the voltage here that is significant.

In contrast to the processes of the present invention, conventional electrolysis processes operate at voltages and/power densities at which HClO⁻ and/or ClO₂ are produced. Indeed, it is these species in conventional electrochemically activated water which is relied upon for the biocidal activity.

A person skilled in the technical field of the present invention would have no difficulty adjusting the parameters of the electrolysis process in order to achieve the voltage and/or power density at the electrodes required to produce the compositions of the present invention, once he is aware of the required voltage and/or power density at the electrodes relevant to the membrane in use.

The ratio of the anolyte:catholyte flow can be from 5:95 to 95:5 with the optimum flow ratio being be 90:10, whilst the current should be between 1 and 20 amps with the preferred range being 6 to 12 amps.

The variation of the chlorine concentration in ppm with water flow at constant current is shown in Table 9.

TABLE 9

| | | | | | Catholyte | |
|---|---|---|---|---|---|---|
| Total Flow | Anolyte | | | | Flow | |
| litres/hr | Flow | Cl (ppm) | O.R.P. | pH | (l/hr) | pH |
| 37.2 | 37.2 | 0 | n/a | 6.78 | 0 | n/a |
| 37.2 | 31.2 | 200 | 1053 | 3.16 | 6 | 12.28 |
| 39 | 27.6 | 250 | 1060 | 3.05 | 11.4 | 12.19 |
| 42 | 24 | 250 | 1069 | 2.87 | 18 | 11.98 |
| 42 | 22.8 | 300 | 1072 | 2.7 | 19.8 | 11.82 |
| 42 | 16.2 | 375 | 1093 | 2.45 | 25.8 | 11.89 |

In addition, the application of a low current during the electrochemical activation process also results in a solution with a reduced chlorine content. This is likely to be due to the fact that the lower magnetic field has a lower ability to attract the negative chloride ions during the brief period that any specific chloride ion is within the electromagnetic field. As a result there is a fat greater probability that the electrolysis will involve the water molecules will which will lie alongside the electrodes and produce oxygen based species than chlorine based species.

That the electrochemically active water produced by the ECA 2000 series machines has an exceptionally high concentration of oxygen is shown in FIG. 4.

In FIG. 4, “Hydroactive” is the electronically activated water anolyte stream from ECA 2000 and “Cell Waste Stream” is the electronically activated water catholyte stream from ECA 2000.

By comparison the normal saturated concentration for oxygen in water is approximately 8.3 mg/litre.

Regardless of the method used to produce the electrochemically activated water of the present invention, the water has the beneficial properties and activity discussed above.

Where the chlorine content of the electrochemically activated water is to be limited by using a low sodium chloride concentration in the water fed into the apparatus carrying out the electrochemical activation, the water fed into the apparatus and electrochemically activated preferably has a sodium chloride concentration of between 1000 and 5000 ppm with the preferred ranges being between 2000 & 3000 ppm chloride ion concentration.

The conventional electrochemical activation processes apply a current of approximately 10-20 amps to the sodium chloride solution. In order to reduce the chlorine content of the electrochemically activated water, it is recommended that the current be reduced to between 1 and 10 amps with the preferred range being between 5 and 9 amps.

The ECA 2000 generator system may be preferably used to generate the electrochemically activated water. This system significantly reduces the formation of undesirable trihalomethane and chloroamine by-products.

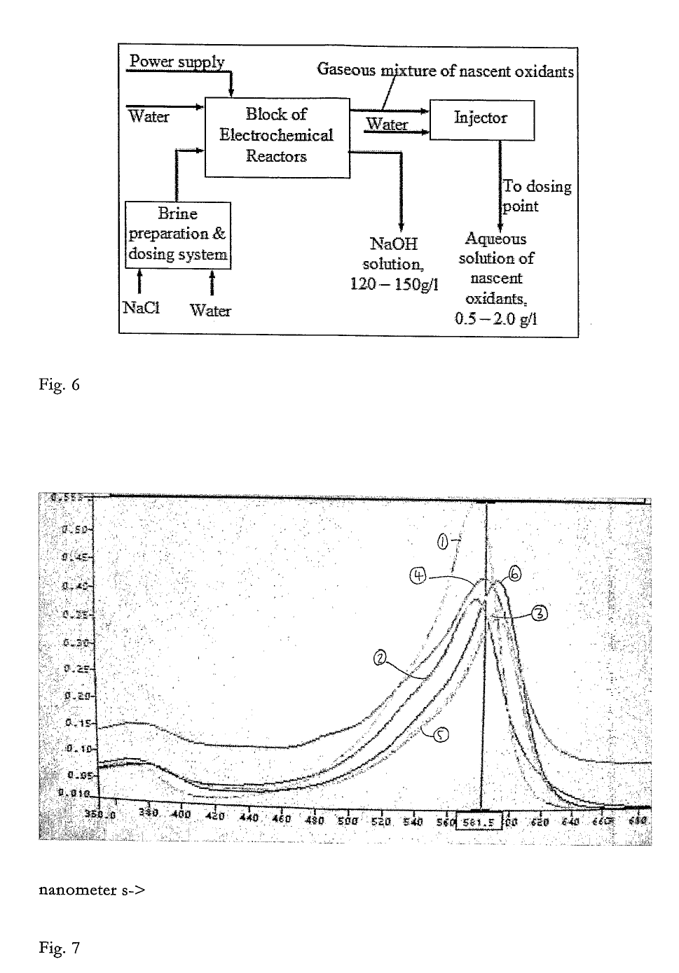

The main principle of ECA 2000 generator devices is an electrochemical synthesis of gaseous mixture of oxidants from a dilute solution of sodium chloride under pressure in diaphragm modular electrochemical elements, each of which is a separate electrochemical rector. The block diagram of such a device is presented on FIG. 6.

As shown on FIG. 6, sodium chloride solution is delivered into the electrochemical reactor. The process results in a partial division of sodium chloride solution into activated chlorine and oxygen based oxidants in the anode chamber, and hydrogen and sodium hydroxide formed in the cathode chamber. The oxidants produced in the anode chamber together with micro-droplets of water are collected (preferably in a darkened acid resistant air tight chemical drum.) The electrochemically active water is then delivered by the injection pump into the part of water to be processed resulting in a dilute solution of oxidants in the final treated water. Hydrogen is generated in the cathode chambers of electrochemical elements and is vented to air through the catholyte discharge tube.

Previous equipment used a ceramic membrane to separate the solutions resulting from close contact with the electrodes. The applicant of the present application has developed the “Flat” core which consists of two half cells working at +15V→0 volts and 0→−15V working back to back where the water to be processed flows across the electrodes and the electrode pairs (and thus the solutions) are separated by an ion exchange membrane. It is this core which can preferably be used to produce the electrochemically active water which is described herein. It is non-ceramic.

The apparatus according to the present invention preferably has a feedback mechanism to maintain a constant current, which is dependent upon the ionic strength of the water, which is in turn dependent upon the salt content and the nature of the water used.

Provided that the storage conditions are suitable, the electrochemically activated water produced according to the present invention can be stored for periods of weeks or months. In a preferred embodiment, the electrochemically activated water can be stored for more than 72 hours. As discussed above, the disinfectant activity of the minimal chlorine electrochemically activated water is due to an active chlorine and oxygen species in the water. It is clear that if gas is allowed to escape from the water during its storage or use, the activity will be diminished or lost. Therefore, it is necessary to ensure that the water is stored in a closed system which will keep the loss of gas from the water to a minimum. Any stirring or other agitation of the water should also be avoided, as this will encourage gas to escape from the water.

When stored in sealed bottles with minimal head space the dissolved active oxygen species will remain in solution above the saturated oxygen in water concentration of approx 8.3 mgs/litre for a considerable period of time. Thus maintaining the activity of the product (see FIG. 2).

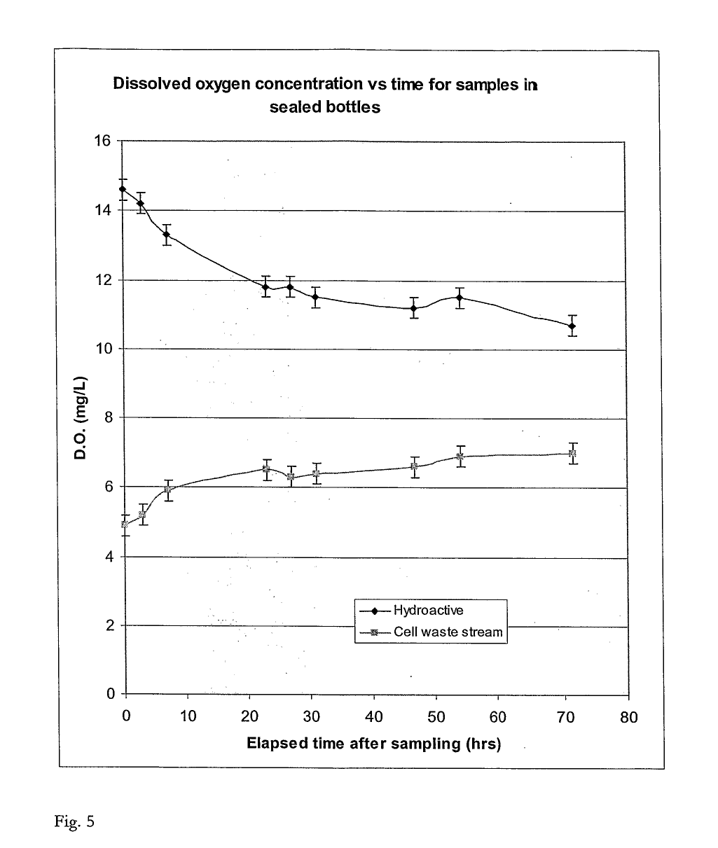

FIG. 5 is a graph showing a three days study of dissolved oxygen concentrations vs time for electronically activated water produced by ECA 2000 Series Generator. The electrochemically activated water solutions of the present invention are ecologically friendly and present no problems for the environment.

This is shown by the concentrations of chlorite (ClO₂), chlorate (ClO₃) and perchlorate (ClO₄), measured in neat freshly prepared solution as recorded in Table 10.

TABLE 10
Concentrations of chlorine species found in neat electronically
activated water as produced in ECA 2000 Series generator

| Species | Formula | EAW Conc | Units |
|---|---|---|---|
| Chloride | Cl | 2600-2800 | mg/kg |
| Hypochlorite | ClO⁻ | 9 | mg/l |
| Chlorite | ClO₂⁻ | <0.2 | mg/l |
| Chlorate | ClO₃⁻ | 0.3-1.2 | mg/l |
| Perchlorate | ClO₄⁻ | 8-13 | mug/l |

This is in stark contrast to conventional biocides based on chlorine, which ate highly oxidative in their action and rely on this property to kill bacterial and viruses. In contrast, it is thought that the mildly oxidative solutions of the present invention act gradually over an extended period of time.

Whilst it is the anolyte of the electrochemically activated water which has the disinfectant activity, a combination of the anolyte and catholyte solutions may also be used in the present invention. The catholyte may be added for its known properties as discussed above, including surfactant properties, or it may be added to adjust the pH of the solution to a desired pH. As mentioned above the anolyte will tend to be acidic in pH and this may be undesirable for some of the proposed uses of the minimal chlorine water EAW disinfectant. One such application is the prevention of fungal infections in farmed fish eggs where for survival of the eggs the pH needs to be above 4 and preferably within the range 5.5→7.5.

Combinations of anolyte and catholyte can form stable products which also maintain their disinfectant activity over long periods of time.

In one embodiment of the present invention, the electrochemically activated water with minimal chlorine content is used as one might use a conventional disinfectant. The water may be sprayed on any surfaces which might be contaminated. Thus, the surfaces which are to be cleaned may be washed or sprayed with the water.

As the water is not harmful, surfaces with which food or drink comes into contact may also be washed or sprayed with the water. One particular advantage of the water according to the present invention is that it has no taint so that it is not necessary for a surface that has been cleaned with the minimal chlorine electrochemically activated water to be rinsed or further cleaned in order to remove any harmful residue, as is generally necessary with conventional disinfectants.

A fine mist of the water can be used to disinfect large areas, such as housing for farm animals and vehicles used to transport them. The water may be used liberally, due to its non-hazardous nature, even where it is used in the vicinity of animals, their food and their drinking water.

The electrochemically activated water with a minimal chlorine content is not harmful if it comes into contact with clothes or skin and it does not produce harmful or unpleasant fumes. The electrochemically activated water may even be used to wash the workers' clothes, shoes or even hands, without causing harm.

In a further embodiment of the invention, electrochemically activated water with a minimal chlorine content may be applied directly to the skin. For example animals, including humans, can be sprayed with the water directly. As the water contains little or no hypochlorite or hydrogen peroxide, it does not cause harm if it comes into contact with the skin and so such direct application is safe.

In another embodiment of the invention, drinking water is dosed with the minimal chlorine water and the drink is then ingested by an animal, including a human. This type of use is particularly useful for treating waterborne infections in livestock, for example chickens. When the minimal chlorine water is ingested as drinking water, at effective concentrations, the water is not harmful.

As indicated above, the electrochemically activated water according to the present invention can be used to dose drinking water. When used in this way, the electrochemically activated water has the further advantage that, in addition to killing micro-organisms in a drinking water supply, it also prevents further contamination. In the past, it has been known to treat drinking water supplies to kill off micro-organisms therein. However, following such conventional treatment, the water is exposed to new microbes and it becomes contaminated and the microbial contamination can reach levels of 10⁵ microbes per ml after a few days. In contrast, where the water is treated by adding a dose of minimal chlorine electrochemically activated water, the water appears to resist recontamination and shows a microbial contamination level of only 1-10 cfu per ml over the same period. It is well documented that the provision of clean drinking water is very important for livestock and many studies have shown that clean drinking water can significantly enhance the growth of chickens.

Further uses of the minimal chlorine content electrochemically activated water include use in cleaning medical instruments and the like. As mentioned above, the minimal chlorine content of the water according to the present invention leaves no taint so that there is no need to rinse the washed articles prior to use.

Another use of the water of the present invention is the cleaning of beer supply pipes or equivalent pipes which require disinfection. Again, the fact that the pipes will not need to be rinsed following their cleaning with the electrochemically activated water with minimal chlorine content is a great advantage, as it will simplify and significantly speed up the cleaning process.

To summarise, the electrochemically activated water according to the present invention can be used in any situation where convention disinfectants have previously been used. In addition, the non-hazardous nature of the water and its low chemical load means that it is also suitable for use in ways that conventional disinfectants often should not, or cannot be used, for example, without rinsing and in direct contact with the skin of animals. Furthermore, the water according to the present invention is also safe to ingest in effective doses, and this is certainly not true of conventional disinfectants. Also, the water according to the present invention is cheap and easy to produce and it is harmless to the environment.

1. A composition comprising electrochemically activated water with a chlorine content of no more than 8 ppm.

2. A composition as claimed in claim 1, wherein the composition is suitable for use as a disinfectant.

3. A composition as claimed in either of the preceding claims, wherein the chlorine content of the water is between 0.1 and 8 ppm and a supersaturated oxygen concentration between 10 and 20 mg/litre.

4. A composition as claimed in either of the preceding claims, wherein the chlorine content of the water is between 0.2 and 6 ppm and a supersaturated oxygen concentration between 11 and 17 mg/litre.

5. A composition as claimed in either of the preceding claims, wherein the chlorine content of the water is between 0.3 and 4 ppm.

6. A composition as claimed in any of the preceding claims wherein said chlorine content is no more than 1 ppm.

7. A composition as claimed in any one of the preceding claims, wherein the composition comprises the anolyte solution of the electrochemically activated water.

8. A composition as claimed in claim 7, wherein the solution further comprises the catholyte solution of the electrochemically activated water.

9. A composition as claimed in any one of the preceding claims, wherein the electrochemically activated water has a redox potential of at least +900 mV.

10. A composition as claimed in claim 9, wherein the electrochemical water includes an active oxygen species in an amount between 13 and 20 mg/l.

11. A composition as claimed in any of the preceding claims, for use in killing or destroying a micro-organism.

12. A composition as claimed in any one of claims 1 to 7, for use in cleaning medical instruments and apparatus.

13. A composition as claimed in any one of claims 1 to 7, for use in cleaning pipes.

14. A composition as claimed in claim 13, wherein the pipes are beer supply pipes.

15. A composition as claimed in any one of claims 1 to 7, for treating a drinking water supply to reduce or remove microbial contaminants.

16. A composition as claimed in claim 15, wherein the treatment reduces the rate of re-contamination of the drinking water compared to water which has been decontaminated by another means.

17. A composition as claimed in any one of claims 1 to 7, for use in therapy.

18. A composition as claimed in claim 17, wherein the composition, wherein the composition is for use in the prevention or treatment of an infection in a subject.

19. A composition as claimed in claim 18, wherein a therapeutically effective dose of the composition is administered orally to the subject.

20. A composition as claimed in claim 19, wherein the composition is administered to the subject prior to, during or after exposure to an infectious micro-organism.

21. A composition as claimed in claim 20, wherein the micro-organism is a virus.

22. A method of killing a micro-organism comprising exposing the micro-organism to a composition as claimed in any one of the preceding claims.

23. Use of a composition as claimed in any one of claims 1 to 7, in the manufacture of a medicament for the treatment or prevention of infection of a subject by an infectious micro-organism, wherein a therapeutically effective dose of the electrochemically activated water is administered to the subject.

24. A use as claimed in claim 23, wherein the composition is administered orally.

25. A method of preparing a composition as claimed in any one of claims 1 to 7, wherein the electrochemically activated water is produced using a sodium chloride-water solution having a chloride ion concentration of 1000-5000 ppm.

26. A method of preparing a composition as claimed in any one of claims 1 to 7, wherein the electrochemically activated water is produced by applying a current of 1-20 amps to a sodium chloride water solution.

27. A method of preparing a composition as claimed in any one of claims 1 to 7, wherein the chlorine content of the electrochemically activated water is reduced to 8 ppm or less by diluting the electrochemically activated water.
